(19) United States
(12) Patent Application Publication
(10) Pub. No.: US 2025/0064336 A1
(43) Pub. Date: Feb. 27, 2025

(54) TISSUE CLASSIFICATION SYSTEM FOR DETECTING CANCEROUS CELLS

(71) Applicant: Johnson & Johnson Enterprise Innovation Inc., New Brunswick, NJ (US)

(72) Inventors: Alexandru Paunescu, Clinton, NJ (US); Charles Scheib, Loveland, OH (US); Ravi Patel, Rochester, NY (US); Aashish Shah, Plymouth, MI (US)

(21) Appl. No.: 18/454,998

(22) Filed: Aug. 24, 2023

(51) Int. Cl. A61B 5/0537; A61B 5/00

(52) U.S. Cl. CPC A61B 5/0537; A61B 5/4839; A61B 5/6848; A61B 5/7264

(57) ABSTRACT

A tissue classification system includes a tube inserted into an anatomy of a patient, with a sensing needle at a distal end of the tube. The sensing needle includes electrodes that generate an electrical field passing through one or more cells of the anatomy contacting one or more electrodes in response to an activation signal. The electrodes generate an electrical sensing signal based on an electrical characteristic of the one or more cells when the electric field is applied. A controller classifies the one or more cells as cancerous or non-cancerous based on the electrical characteristic. Based on the classification, the controller may control a medicament delivery device to enable medicament delivery when the sensing needle meets position criteria relative to a target tumor.

### BACKGROUND

### Technical Field

The described embodiments relate to a system and a method for detecting cancerous cells from electrical measurements of the tissue.

### Description of the Related Art

Conventionally, diagnosis of cancer often begins with detection of nodules or masses from X-ray images or computerized tomography (CT) images of the patient. However, these techniques are unable to directly confirm whether the detected masses or nodules are cancerous or benign. When a suspicious mass or nodule is detected, tissue is conventionally biopsied and analyzed. Biopsies of masses that are insufficient in size may result in inconclusive determinations. Therefore, biopsies may often be delayed while masses are monitored for growth. The delay in potential treatment can negatively impact a patient's likelihood of survival if the mass is determined to be cancerous.

### BRIEF DESCRIPTION OF THE DRAWINGS

FIG. 1 is an example embodiment of a medical environment in which a tissue classification system operates.

FIG. 2 is a diagram of one embodiment of a tissue classification system.

FIG. 3A illustrates a first example use case of a tissue classification system.

FIG. 3B illustrates a second example use case of a tissue classification system.

FIG. 4 illustrates a third example use case of a tissue classification system.

FIG. 5 is a block diagram of one embodiment of a sensing configured to receive an optical activation signal and to transmit an optical sensing signal.

FIG. 6 is a flowchart illustrating an example embodiment of a process for controlling medicament delivery based on tissue classification.

FIG. 7 is a flowchart illustrating an example embodiment of operating a tissue classification system using optical communication.

FIG. 8 is a first example embodiment a probe tip for sensing electrical characteristics of cells for tissue classification with an expandable substrate.

FIG. 9 is a second example embodiment a probe tip for sensing electrical characteristics of cells for tissue classification with an expandable substrate.

FIG. 10 is a third example embodiment a probe tip for sensing electrical characteristics of cells for tissue classification with an expandable substrate.

FIG. 11 is a flowchart of an example embodiment of a method for classifying one or more cells as cancerous or non-cancerous using a probe tip with an expandable substrate.

### SUMMARY

A tissue classification system controls medicament delivery based on classification of tissue from electrical characteristics sensed by a sensing needle. The tissue classification system includes a tube for inserting into an anatomy and a sensing needle at a distal end of the tube. The sensing needle includes at least a first set of electrodes that are selectively activated in response to an activation signal to generate a first electric field that passes through a first group of cells of the anatomy. The first set of electrodes generate a first sensing signal representing a sensed electrical characteristic of the first group of cells in response to the first electric field. A controller is configured to apply the activation signal, to receive the first sensing signal, to obtain a first classification of the first group of cells as cancerous or non-cancerous based on the sensed electrical characteristic in the first sensing signal, and to control medicament delivery via the sensing needle based on at least the first classification.

In an embodiment, the tissue classification system further includes at least a second set of electrodes to generate a second electric field that passes through a second group of cells of the anatomy, The second set of electrodes generates a second sensing signal. The controller furthermore obtains a second classification of the second group of cells based on the second sensing signal, and controls the medicament delivery further based on the second classification.

In an embodiment, the first set of electrodes and the second set of electrodes comprise respective radial arrays of electrodes at different distances from a tip of the sensing needle. Here, the controller may control the medicament delivery by enabling a medicament delivery pump responsive to the first classification and the second classification being indicative of the sensing needle penetrating a target tumor with a predefined penetration depth.

In an embodiment, the first set of electrodes and the second set of electrodes are radially spaced on a surface of the sensing needle. Here, the controller controls the medicament delivery by enabling a medicament delivery pump responsive to both the first classification and second classification classifying the first group of cells and the second group of cells respectively as cancerous.

In an embodiment, the controller activates a lockout mechanism of a medicament delivery pump to prevent the medicament delivery via the sensing needle in absence of a delivery criteria based at least in part on the first classification being met.

In an embodiment, the controller obtains the first classification by determining a Cole relaxation frequency from the sensed electrical characteristic of the first group of cells, and classifying the first group of cells as cancerous in response to the Cole relaxation frequency being within a specified range of frequencies or classifying the first group of cells as non-cancerous in response to the Cole relaxation frequency being outside the specified range of frequencies.

In an embodiment, the controller is configured to generate the activation signal as an optical activation signal and to receive the first sensing signal as an optical sensing signal. In this embodiment, the sensing needle further comprises an optical-to-electrical converter to convert the optical activation signal to an electrical activation signal applied to the first set of electrodes, and an electrical-to-optical converter to convert an electrical sensing signal to the optical sensing signal for outputting to the controller. Furthermore, the tube comprises a fiber optic link for communicating the optical activation signal and the optical sensing signal.

In an embodiment, the fiber optic link includes a first fiber optic channel for communicating the optical activation signal, and a second fiber optic channel for communicating the optical sensing signal.

In another embodiment, the fiber optic link includes a shared fiber optic channel for bidirectionally communicating the optical activation signal and the optical sensing signal.

In another aspect, a method controls medicament delivery based on tissue classification. A first activation signal is generated to activate at least a first set of electrodes of a sensing needle to cause the first set of electrodes to generate a first electric field that passes through a first group of cells of an anatomy. A controller obtains, from the first set of electrodes, a first sensing signal representing a sensed electrical characteristic of the first group of cells in response to the first electric field. The controller obtains, based on the first sensing signal, a first classification of the first group of cells as cancerous or non-cancerous, and determines if the sensing needle meets position criteria relative to a target tumor based at least in part on the first classification. The controller controls a medicament delivery system to enable medicament delivery via the sensing needle in response to the sensing needle meeting the position criteria.

In an embodiment, controlling the medicament delivery system comprises enabling a medicament delivery pump. In a further embodiment, the medicament delivery pump is locked responsive to the sensing needle not meeting the position criteria.

In an embodiment, the method further comprises generating a second activation signal to activate at least a second set of electrodes of the sensing needle to cause the second set of electrodes to generate a second electric field that passes through a second group of cells of the anatomy. The controller obtains, from the second set of electrodes, a second sensing signal representing a sensed electrical characteristic of the second group of cells in response to a second electric field, and obtains, based on the second sensing signal, a second classification of the second group of cells as cancerous or non-cancerous. The controller determines if the sensing needle meets position criteria relative to a target tumor based at least in part on the second classification.

In an embodiment, the first set of electrodes and the second set of electrodes comprise respective radial arrays of electrodes at different distances from a tip of the sensing needle. In this embodiment, the controller may determine if the sensing needle meets the position criteria by determining, based on at least the first classification and the second classification, that the sensing needle achieves at least a predefined penetration depth into the target tumor.

In an embodiment, the first set of electrodes and the second set of electrodes are radially spaced on a surface of the sensing needle. The controller may determine if the sensing needle meets the position criteria by determining, based on at least the first classification and the second classification, that the sensing needle is fully surrounded by the target tumor.

In an embodiment, obtaining the first classification comprises determining a Cole relaxation frequency from the sensed electrical characteristic of the first group of cells, and classifying the first group of cells as cancerous in response to the Cole relaxation frequency being within a specified range of frequencies or classifying the first group of cells as non-cancerous in response to the Cole relaxation frequency being outside the specified range of frequencies.

In an embodiment, generating the first activation signal comprises generating an optical activation signal at a controller, transmitting the optical activation signal over a fiber optic link coupled between the controller and the sensing needle, and converting the optical activation signal to an electrical activation signal by an optical-to-electrical converter of the sensing needle. Furthermore, obtaining the first sensing signal comprises generating an optical sensing signal by an electrical-to-optical converter of the sensing needle based on an electrical sensing signal, transmitting the optical sensing signal over the fiber optic link from the sensing needle to the controller.

In another aspect, a medical targeting system controls medicament delivery to a target tumor based on tissue classification. The medical targeting system includes at least an imaging system comprising an endoscope for capturing images of an anatomy, and a tissue classification system for controlling medicament delivery as described above. The medical targeting system may furthermore comprise a robotic guidance system to control navigation of at least one of the endoscope and the sensing needle.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

Animal cells are surrounded by a membrane comprising a lipid bilayer with proteins embedded in the lipid bilayer membrane, with the cell membrane acting as an insulator and a diffusion barrier to movement of ions between an interior environment and an exterior environment of the cell. Transmembrane proteins (e.g., ion transporter proteins or ion pump proteins) actively push ions across the cell membrane to establish concentration gradients across the cell membrane. Ion channels in the cell membrane allow ions to move across the cell membrane via the concentration gradients. Ion pumps and ion channels are electrically similar to a set of batteries and resistors inserted in the cell membrane, creating a voltage between an interior side and an exterior side of the cell membrane. Consequently, a cell membrane behaves similarly to a leaky electrical capacitor.

A cell membrane may be modeled as a resistor-capacitor (RC) circuit, with this model allowing evaluation of electrical characteristics of a cell. For example, application of an alternating current (AC) electric field to a cell allows detection of one or more electrical characteristics of the cell, such as impedance. Studies have shown that cancerous and non-cancerous cells tend to have differing electrical characteristics. In an example technique for characterizing a likelihood of a cell being cancerous, a measured impedance for a cell is converted into a Cole relaxation frequency (CRF). The CRF for the cell uses a characteristic time for charge applied to the cell to redistribute, which is based on a product of an internal resistance of the cell's membrane and a capacitance of the cell's membrane. The CRF for a cell is correlated with a likelihood of the cell being cancerous or being non-cancerous. In various embodiments, a peak value of a CRF for a cell determines a likelihood of the cell being cancerous or non-cancerous. For example, in one estimation technique, a cell with a peak value of its CRF in a specific range (e.g., 0.6 MHz to 2 MHz) is determined to be cancerous, while a cell with a peak value of its CRF outside of the specific range is determined to be non-cancerous. The above techniques may be applied to detect different types of cancers including, for example, breast cancer, lung cancer, colon cancer, pancreatic cancer, skin cancer, or other types of cancer.

The disclosed embodiments include a device for sensing electrical characteristics and classifying cells as cancerous or non-cancerous. The device may include a thin flexible or rigid tube with a sensors at the distal end. In some embodiments, the sensors may be integrated with a needle that may deliver a medicament to a target anatomy. Alternatively, the sensors may comprise a standalone sensing probe tip that is not necessarily integrated with a needle for drug delivery. The sensors includes electrodes that may be activated by a control signal to generate an electrical field and sense electrical characteristics of cells in proximity to the electrodes. The device may be inserted into an anatomical channel, such as a trachea or bronchial tubes, or may be inserted through tissue to a target site. The activated electrodes sense an electrical characteristic such as an impedance. A controller classifies cells proximate to the sensors as cancerous or non-cancerous based on the sensed electrical characteristic. For example, the controller determines a Cole relaxation frequency (CRF) based on the sensed electrical characteristic, and classifies cells as cancerous or non-cancerous based on the determined CRF. The device may enable selectively activate or deactivate differently positioned electrodes to vary the electrical field and enable various measurements characterizing the tissue proximate to a set of spaced sensors.

In an embodiment, where the sensors are integrated into a sensing needle capable of medicament delivery, the sensing needle may be inserted into tissue to detect position of the sensing needle relative to a target site such as a tumor. The sensing needle may have multiple radially spaced electrodes in a set of radial arrays at different distances from the needle tip that may each independently detect cancerous cells. In this configuration, by analyzing the sensing signals from different electrodes and different positions along the sensing needle, a controller may detect when the needle is fully inserted into a tumor and may furthermore estimate the insertion depth. When the needle is inserted at a depth appropriate for medicament delivery, the device may enable a delivery pump to deliver medicament to the target cells.

To enable miniaturization and reduce parasitic effects of the connection between the sensors and an external controller, the tube may include one or more fiber optic channels for coupling the controller to the sensing needle. In this configuration, the controller generates an optical activation signal that is transmitted to the sensing needle through the fiber optic channel of the tube. An optical-to-electrical converter in the sensing needle converts the optical activation signal to an electrical activation signal that is directed to at least a subset of the electrodes to selectively generate an electric field. The electrodes generate an electrical sensing signal representing sensed electrical characteristics when the electric field is activated. An electrical-to-optical converter included in the sensing needle converts the electrical sensing signal to an optical sensing signal that is sent to the controller via the fiber optic channel(s). This configuration beneficially enables a probe tip that is small enough to traverse the anatomical channels without the signals being substantially degraded by parasitic effects of the connection.

In another embodiment, the sensors may be integrated into a sensing probe tip that does not necessarily include a needle for medicament delivery. In some such embodiments, the sensing probe tip may include an expandable substrate and a set of electrodes on the expandable substrate. The expandable substrate and the set of electrodes are on an external surface of a balloon (or other expandable device), with the balloon configured to be inflated to maintain conformal contact (or reduce gaps) between the electrodes and the surrounding cells. In various embodiments, the set of electrodes are positioned on the expandable substrate so that a distance between adjacent electrodes remain substantially fixed independently of the inflation state of the balloon. Alternatively, the electrodes may comprise an expandable conductive material whose conductive characteristics are not altered when the conductive material is expanded. Improving contact between the sensing probe tip and the target tissue effects may mitigate the effects of noise and improve classification performance.

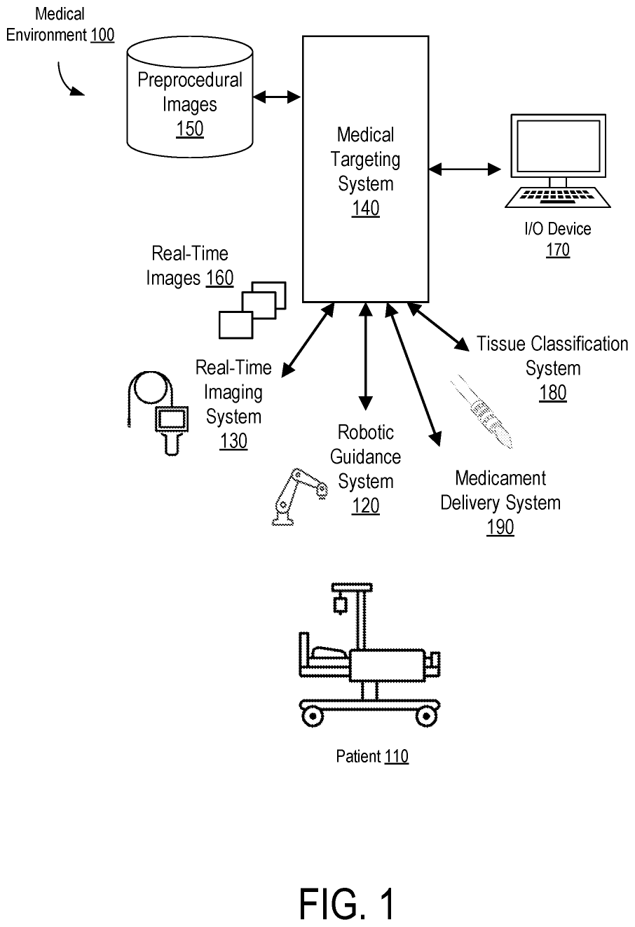

FIG. 1 illustrates an example embodiment of a medical environment 100 for classifying tissue of a patient 110 and optionally delivering medicament to target tissue based on the classification. The medical environment 100 includes a medical targeting system 140, a robotic guidance system 120, a real-time imaging system 130 for capturing real-time images 160, a tissue classification system 180, a medicament delivery system 190, a preprocedural image datastore storing preprocedural images 150, and one or more input/output (I/O) devices 170. The tissue classification system 180 and medicament delivery system 190 may be integrated together in some embodiments, for example, as a medicament delivery needle with integrated sensors. Alternatively, the tissue classification system 180 and medicament delivery system 190 may comprise independent devices. In different variations, the medical environment 100 may include different or additional components.

The medical targeting system 140 and preprocedural image datastore storing the preprocedural images 150 may be all or partially co-located with the patient 110 (e.g., in an operating or examination room of a medical facility), or may be at least partially located remotely (e.g., in a server room of a medical facility or in a cloud server system remote from the medical facility). The various electronic components 120, 130, 140, 150, 170 may all or partly be communicatively coupled via one or more networks (e.g., a local area network (LAN), wide area network (WAN), cellular data network, or combination thereof), and/or may be directly communicatively coupled via wired or wireless communication links (e.g., WiFi direct, Bluetooth, Universal Serial Bus (USB), or other communication link).

The preprocedural images 150 comprise a set of images of the patient 110 captured prior to the medical procedure. The preprocedural images 150 may comprise, for example, CT scan images, magnetic resonance imaging (MRI) images, ultrasound images, X-ray images, positron emission tomography (PET) images, or other images or combinations thereof relevant to the procedure. The preprocedural images 150 may be annotated to indicate one or more target locations associated with an anatomical target (e.g., a tumor or other nodule, mass, lesion, or other anatomical structure) of the procedure.

The real-time imaging system 130 captures real-time images 160 of the patient 110 during the medical procedure. The real-time imaging system 130 may comprise an endoscope having one or more light sources and one or more light sensors (e.g., cameras) coupled to a probe tip of a long, thin, tube (e.g., rigid or flexible) that can be threaded through various anatomical channels such as airways, blood vessels, gastrointestinal tract, or other channels, or other pathways (such as through tissue) including those that may be formed by a needle, cannula, or other instrument. The real-time imaging system 130 may furthermore include one or more sensors (e.g., an electromagnetic coil) proximate to the probe tip that enables a location-sensing system to detect a location of the probe tip as it traverses through the anatomy.

The probe tip of the real-time imaging system 130 may comprise a camera (e.g., single camera, stereoscopic camera, or multi-view camera) that captures images of the anatomy. The endoscope could also have one or more working channels that enable one or more instruments, such as a sensing needle or other sensing probe of the tissue classification system 180, to pass through the endoscope to a position proximate to the camera.

The tissue classification system 180 senses characteristics of tissue and classifies the tissue as cancerous or non-cancerous based on the sensed characteristics. The tissue classification system 180 may include a lumen structured to pass through the working channel of the real-time imaging system 130 described above such that sensors at a distal end are positioned proximate to the probe tip of the real-time imaging system 130. The sensing device of the tissue classification system 180 may optionally be physically coupled to the real-time imaging system 130 (e.g., in an integrated device) such that it navigates to a desired location together with the real-time imaging system. Alternatively, the tissue classification system 180 may comprise a standalone device that is not necessarily used in conjunction with a real-time imaging system 130 and may be independently navigated to a desired anatomical location using a navigation system that is not necessarily reliant on real-time images.

The medicament delivery system 190 may comprise a pump and various control mechanisms for controlling dispensing of the medicament. The medicament delivery system 190 may optionally be integrated with the tissue classification system 180 as a delivery needle with integrated sensors for tissue classification and a controller for controlling sensor activation, classification, and medicament delivery. Alternatively, the medicament delivery system 190 may comprise a standalone medicament delivery device separate from the tissue classification system 180.

The robotic guidance system 120 facilitates movement of the real-time imaging system 130 through one or more anatomical channels or other pathways of the patient 110 (e.g., through tissue). The robotic guidance system 120 may comprise a robotic-assisted device that facilitates control and navigation of the real-time imaging system 130 and/or the tissue classification system 180 responsive to control inputs provided by a medical professional using a I/O device 170 (e.g., a handheld controller or other computer input device). Alternatively, the robotic guidance system 120 may provide automated guidance in which it directly navigates the real-time imaging system 130 and/or the tissue classification system 180 to the anatomical target without necessarily requiring manual control inputs. Here, the robotic guidance system 120 may facilitate navigation based on a predefined pathway or the robotic guidance system 120 may automatically determine the pathway.

The robotic guidance system 120 may enable real-time tracking of the probe tip of the real-time imaging system 130 and/or the tissue classification system 180 using electromagnetic tracking, image-based tracking (including white light and/or fluorescent light tracking), shape sensing, or other techniques. The robotic guidance system 120 may furthermore control actuation of the tissue classification system 180 (e.g., application of an activation signal to the tissue classification system 180).

In different embodiments, the robotic guidance system 120 may operate with different levels of autonomy. For example, in some embodiments, the robotic guidance system 120 is entirely under control of a human operator that controls movements using the I/O device 170. In other embodiments, the robotic guidance system 120 may operate in conjunction with the medical targeting system 140 to perform completely automated procedures based on the target marked in the preprocedural images 150. Other embodiments may include a combination of manually controlled and automatically controlled functions.

The medical targeting system 140 performs a registration between the set of preprocedural images 150 and the real-time images 160 to map between respective coordinates of the image spaces in the preprocedural images 150 and the real-time images 160. In this way, the real-time position of the probe end of the real-time imaging system 130 can be mapped to a specific position in the image space of the preprocedural images 150. Furthermore, the target position of the anatomical target in the preprocedural images can be mapped to a virtual target location in the real-time images. A location of sensing tip of the tissue classification system 180 may similarly be registered to the real-time images 160 and/or the preprocedural images based on predefined physical alignment between the tissue classification system 180 and the probe tip of the real-time imaging system 130, electromagnetic tracking, image-based tracking, or a combination thereof.

The medical targeting system 140 may furthermore determine a navigation path to a target position in the preprocedural images 150. For example, the medical targeting system 140 may generate control signals for controlling navigation of the real-time imaging system 130 to a vicinity of the target. The tissue classification system 180 may then be deployed to classify tissue at the virtual target position as cancerous or non-cancerous, as further described below. The tissue classification system 180 may detect, based on the classification of cells, when an integrated medicament delivery needle is positioned within a tumor at sufficient depth to enable medicament delivery and then enable delivery of a medicament (e.g., by activating a delivery pump of the medicament delivery system 190) when the delivery needle is appropriately positioned within a target tumor. Alternatively, in embodiments where the medicament delivery device is separate from the tissue classification system 180 may be withdrawn once the cancerous tissue is detected, and a separate medicament delivery device may then be deployed to the target location.

The medical targeting system 140 may be implemented using on-site computing and/or storage systems, cloud computing and/or storage systems, or a combination thereof. Accordingly, the medical targeting system 140 may be local, remote, and/or distributed with portions being local and portions remote, where the various system elements may be communicatively coupled over a network. The medical targeting system 140 may implement the functions described herein by one or more processor and a non-transitory computer-readable storage medium that stores instructions executable by the one or more processors to perform the described functions.

The I/O device 170 may render various views of the preprocedural images 150 and/or the real-time images 160 to facilitate the medical procedure. Various virtual objects and/or other data may optionally be overlaid on the preprocedural images 150 and/or the real-time images 160. For example, a location of the target marked in the preprocedural images and registered to the real-time images 160 may be rendered as a virtual object in a display of the real-time images 160. Furthermore, a tracked location of the probe tip of the real-time imaging system 130 or a sensing needle of the tissue classification system 180 may be mapped to coordinates in the preprocedural images 150 and overload on the preprocedural images 150 to track movement of the real-time imaging system 130 and/or the tissue classification system 180. Classification data derived from the tissue classification system 180 may furthermore be overlaid on the real-time images 160 and/or the preprocedural images 150 to provide real-time information about the probed tissue. For example, the classification data may indicate likelihoods of tissue being cancerous and/or non-cancerous or may outline in the images areas of tissue having high likelihoods of being cancerous.

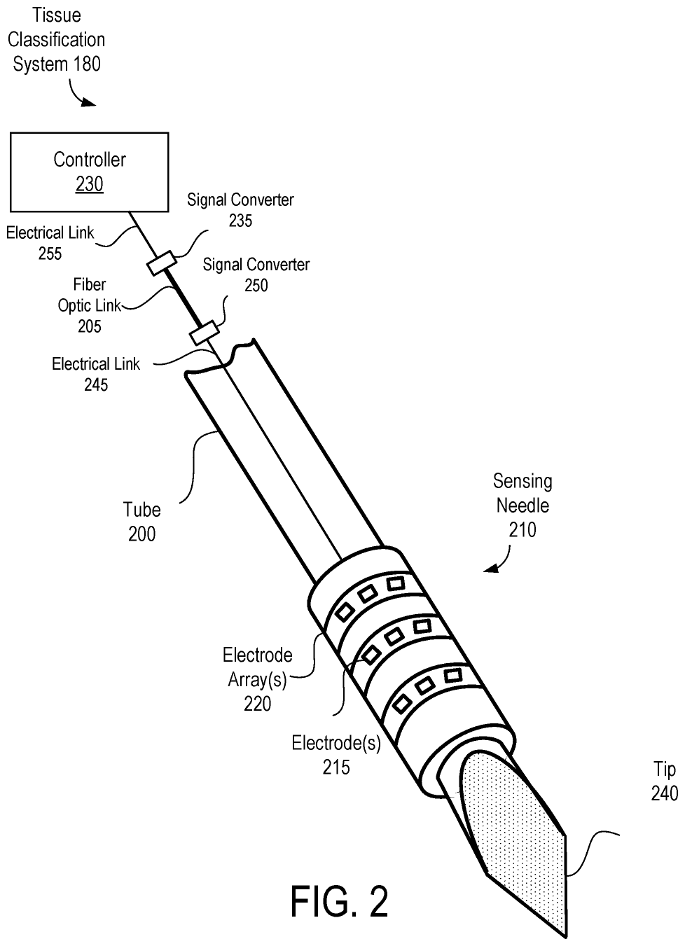

FIG. 2 is a diagram of one embodiment of a tissue classification system 180. In the example shown by FIG. 2, the tissue classification system 180 includes a tube 200, a sensing needle 210 (including a tip 240, and one or more electrode arrays 220 each comprising one or more electrodes 215), and a controller 230. In other embodiments, the tissue classification system 180 includes additional or different components. Further, in some embodiments, functionality provided by multiple components shown in FIG. 2 may be combined, so the tissue classification system 180 may include fewer components than those described in conjunction with FIG. 2.

In various embodiments, the tissue classification system 180 comprises a tube 200 with the sensing needle 210 at a distal end of the tube 200. The tube 200 is configured to be inserted into an anatomy of a patient to direct the sensing needle 210 to a desired location. In an embodiment structured for lung cancer detection and treatment, the tube 200 and the sensing needle 210 may be structured for inserting into an anatomical channel of a patient, such as a trachea or bronchial tube of the patient. In other embodiments, the tube 200 and the sensing needle 210 may be inserted through tissue and not necessarily through an existing anatomical channel. In some embodiments, the tube 200 and the sensing needle 210 are inserted through a working channel of an endoscope, as further described above in conjunction with FIG. 1. The tube 200 is advanced through an anatomical channel or a working channel of an endoscope so the sensing needle 210 directly contacts (or is closely proximate to) one or more cells. In various embodiments, a robotic guidance system 120 moves the sensing needle 210 through the anatomy of the patient based on real-time images 160 from a real-time imaging system 130 and preprocedural images 150 of the anatomy, as further described above in conjunction with FIG. 1. Here, the sensing needle 210 may include various electronics associated with enabling location tracking and/or navigation of the sensing needle 210. In various embodiments, the tube 200 is a rigid material, while in other embodiments the tube 200 is a flexible material.

The tissue classification system 180 may furthermore include a fiber optic link 205 comprising one or more fiber optic channels. A controller-side signal converter 235 may convert between electrical activation signals originating from the controller 230 via an electrical link 255 and optical activation signals transmitted over the fiber optic link 205. The controller-side signal converter 235 may furthermore convert between optical sensing signals communicated via the fiber optic link 205 and electrical sensing signals provided to the controller 230 via the electrical link 255. A sensing needle-side signal converter 250 may similarly convert between the optical activation signals received from the fiber optic link 205 and electrical activation signals for providing to the sensing needle 210 via an electrical link 245, and convert between electrical activation signals originating from the sensing needle 210 (via the electrical link 245) and optical sensing signals communicated via the fiber optic link 205. In various embodiments, the sensing needle-side signal converter 250 may be integrated with the sensing needle 210 or may comprise a separate device within the tube 200 or externally to the tube 200. Furthermore, the controller-side signal converter 235 may be integrated with the controller 230 or may comprise a separate device.

The optical activation signal and the optical sensing signal may have different wavelengths, allowing both optical signals to be transmitted through a single fiber optic channel of the fiber optic link 205 without interfering with each other. Alternatively, the signals may be multiplexed based on time, codes, phases, or other multiplexing techniques. In embodiments where the fiber optic link 205 includes multiple fiber optic channels, one fiber optic channel may receive the optical activation signal from the controller 230 and a different fiber optic channel may receive the optical sensing signal from the sensing needle 210. In some embodiments, the fiber optic link 205 includes additional fiber optic channels for transmission of other signals.

The sensing needle 210 includes a set of electrodes 215 coupled to a surface of the sensing needle 210. In various embodiments, the electrodes 215 may be arranged in two or more arrays 220 organized in radial bands with the electrodes 215 separated by non-conducting material. Alternatively, the electrodes 215 may be organized in other array configurations such as longitudinal columns of electrodes 215 or localized sub-matrices of electrodes 215. In some embodiments, the electrodes 215 within an array 220 may have substantially fixed spacing in at least one direction. For example, in the radially oriented electrode arrays 220 of FIG. 2, the electrodes 215 in each array may have substantially fixed radial spacing. Spacing between electrode arrays 220 (e.g., longitudinal spacing in FIG. 2) may also be substantially uniform or arrays 220 may have non-uniform spacing. For example, a sensing needle 210 may have a set of five electrode arrays 220 in radial bands spaced approximately 5 mm apart, with each electrode array 220 including a set of six sensing electrodes 215 spaced 60 degrees apart. In another embodiment, electrodes 215 may be located in the tip 240 of the sensing needle 210. For example, one embodiment may include a sensing needle 210 with a single electrode array 220 as a radial ring around the tip 240.

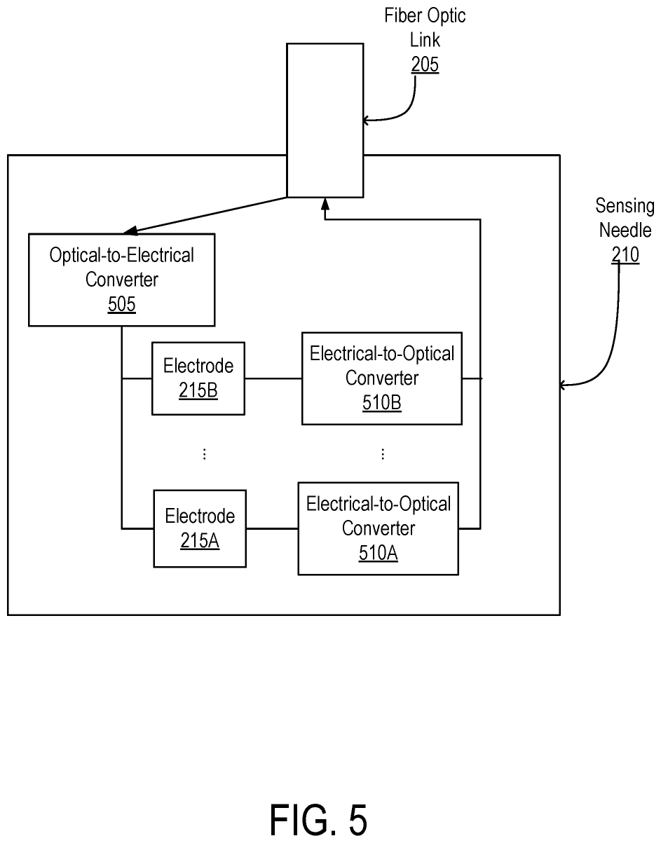

The sensing needle 210 may furthermore include various other electronics and/or optical components that may be coupled to the electrodes 215 as described further in FIG. 5. In one construction, a needle may comprise a shaft having a metal base material with a non-conducting material (such as plastic) that may be epoxied, over-molded, or interference fit to the surface. One or more metal (e.g., copper) rings may similarly be epoxied, over-molded, or interference fit to the plastic to form the sensors. Wiring from the electrodes 215 to other electronics may be achieved using, for example, a flex circuit, wires embedded within walls of the needle shaft using co-extrusion construction, or various 3D printing techniques.

The controller 230 is coupled to each electrode 215 and is configured to communicate an activation signal to at least a subset of the electrodes 215. The activation signal causes electrodes 215 receiving the activation signal to generate an electric field between the electrodes 215. The generated electric field passes through one or more cells of the anatomy that are contacting or closely proximate to the electrodes 215 of the subset.

The controller 230 may selectively activate different combinations of electrodes 215 to shape the electric field. For example, by activating different electrodes, the sensing needle 210 may generate electric fields of varying depth of penetration, varying shape, and varying levels of localization. The distance between activated electrodes 215 may affect properties of the generated electric field. For example, activating electrodes 215 that are closer together generates an electrical field with reduced penetration depth relative to an electrical field generated by electrodes 215 that are further apart. The controller 230 may actively select different pairs of electrodes 215 or electrode arrays 220 in order to control the depth and level of localization. For example, the controller 230 may select electrodes 215 relatively far apart to generate a relatively wide electrical field and measure electrical characteristics of cells across a relatively wide area, or may select electrodes 215 closer together to generate a relatively narrower electrical field and measure electrical characteristics of cells across a relatively narrower area. In various embodiments, a medical professional or other user may selectively control the depth of penetration and probed area by causing the controller 230 to selectively activate different combinations of electrodes 215.

In some embodiments, the controller 230 may generate different types of activation signals that cause generation of one or more fields having different characteristics. Generation of differently behaving fields may modulate cell membrane ion channel behavior of cells of the anatomy differently and may thereby increase a specificity with which different cells of the anatomy may be identified as cancerous or non-cancerous. For example, the controller 230 may generate activation signals that cause one or more of an alternating current (AC) electric field, a direct current (DC) electric field, or another electric field varying more slowly than the AC electric field. Different types of activation signal may be applied sequentially or concurrently to different pairs of electrodes 215. Directionality of the DC electric field, or other more slowly varying electric field, may result in measurements that identify an anisotropy of the cells and can provide additional measurements useful for classification of the cells as non-cancerous or cancerous tissue, or about a stage of development of cancerous cells.

Alternatively or additionally, the controller 230 may generate an activation signal that causes selected electrodes 215 to generate one or more magnetic fields, where gradients of the one or more magnetic fields may modulate the resistance of the ion channels of cells by introducing Lorentz force-based deflections. In other embodiments, the controller 230 may enable selected electrodes 215 to generate an ultrasonic field capable of inducing Brillouin scattering in various cells that interferes (constructively or destructively) with impedance of cells to which the electric field is applied. These additional activation signals may enable further measurements of electrical characteristics that provide additional information for classifying cells as cancerous or non-cancerous.

The controller 230 is further configured to receive a sensing signal from the sensing needle 210 (e.g., via the fiber optic link 205 and respective converters 235, 250) that represents measured electrical characteristics of the tissue proximate to the sensing needle 210. For example, the sensed electrical characteristics may represent a sensed impedance, resistance, capacitance, or other sensed characteristic from which the CRF or other signals can be derived. Based on the electrical characteristic, the controller 230 classifies the one or more cells proximate to the sensing needle 210 as cancerous or non-cancerous. In various embodiments, the controller 230 determines a Cole relaxation frequency of the one or more cells based on the sensed electrical characteristic and compares the Cole relaxation frequency to a specific range of frequencies. If the Cole relaxation frequency is within the specific range, the controller 230 classifies the cells as cancerous, while the controller 230 classifies the cells as non-cancerous in response to the Cole relaxation frequency being outside of the specific range. In other embodiments, the controller 230 classifies the one or more cells as cancerous if the Cole relaxation frequency exceeds a threshold and classifies the one or more cells as non-cancerous in response to the Cole relaxation frequency does not exceed the threshold. In various embodiments, the controller 230 determines the Cole relaxation frequency based on the measured impedance and a Cole-Cole impedance model that models cells of an anatomy as a resistor-capacitor circuit, as further described above. For example, the controller 230 determines a value of the Cole relaxation frequency for the Cole-Cole impedance model that has a maximum correlation to the sensed impedance and classifies the one or more cells based on the determined value of the Cole relaxation frequency. In other embodiments, the controller 230 compares the electrical characteristic sensed by the electrodes 215 to one or more other criteria to classify cells that contact the electrodes 215 as cancerous or non-cancerous.

The controller may comprise a dedicated control device or may be implemented in a general computing system. In one embodiment, the controller 230 includes one or more processors and a non-transitory computer-readable storage medium storing instructions associated with the various sensor control functions and classification functions described herein. Different functions of the controller 230 may be performed by a device locally coupled to the sensing needle 210, or may be performed by a network-coupled computing device (such as in a cloud computing environment). The controller 230 may alternatively, or additionally, include an interface for wired or wireless connectivity to the medical targeting system 140. In this embodiment, various control and classification functions may be performed by the medical targeting system 140 and communicated, as relevant, to the controller 230.

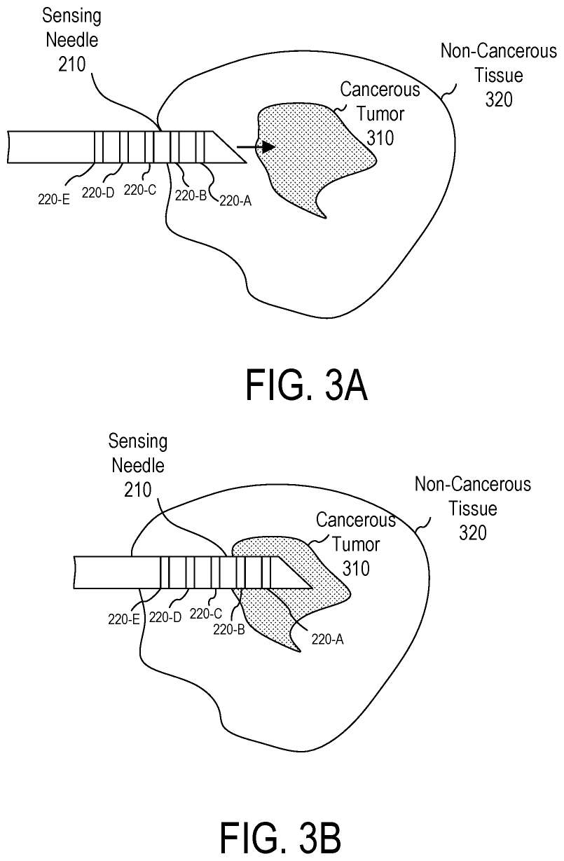

FIGS. 3A-B illustrates an example use case associated with operation of the sensing needle 210 in association with a medical procedure involving delivery of a medicament to a cancerous tumor 310. FIG. 3A illustrates an anatomy including non-cancerous tissue 320 and the cancerous tumor 310. In the position of FIG. 3A, two of the sensor arrays 220-A, 220-B are positioned proximate to the non-cancerous tissue 320 outside of the cancerous tumor 310. In this position, the tissue classification system 180 may classify the tissue around sensor arrays 220-A, 220-B as non-cancerous, thus indicating that the sensing needle 210 is not sufficiently positioned for medicament delivery to the cancerous tumor 310. In this position, the controller 230 may prevent medicament delivery (e.g., by locking a pump mechanism). In FIG. 3B, sensor arrays 220-A, 220-B detect cancerous cells of the cancerous tumor 310 while sensor arrays 220-C, 220-D, 220-E detect non-cancerous tissue 320. Based on the dimensions of the sensing needle 210 and the spacing between sensor arrays 220, the controller 330 can estimate the depth of penetration of the sensing needle 210 into the cancerous tumor 310. The controller 330 may then determine whether or not to activate the medicament delivery system 190 based on the penetration depth to inject medicament into the tumor 310. When predefined position criteria is met, the controller 330 may automatically enable medicament delivery or may disable a locking mechanism of the delivery pump to enable the medicament delivery to be manually activated.

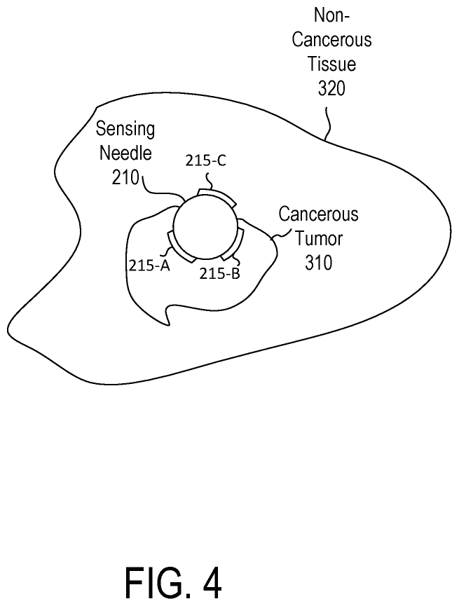

FIG. 4 illustrates another example position of a sensing needle 210 that includes radially distributed electrodes 215. In this example, the sensing needle 210 has only partially penetrated the cancerous tumor 310, but is not completely surrounded by it. In this position, signals from electrode 215-C will be classified as detecting non-cancerous tissue 320 while signals from electrode 215-A, 215-B will be classified as detecting the cancerous tumor 310. In this detected position, the controller 330 may prevent activation of the medicament delivery system 190 because the sensing needle 210 has not fully penetrated the tumor 310. The controller 330 may furthermore generate navigation signals (for a human operate or robotic guidance system 120 to guide the sensing needle 210 towards a position where it fully penetrates the cancerous tumor 310 and medicament can be delivered. In an embodiment, the controller 230 enables medicament delivery only when all of the sensors in at least one radial band detect cancerous cells (i.e., when the sensing needle 210 is fully surrounded by the target tumor).

FIG. 5 is a block diagram of a sensing needle 210. The sensing needle 210 includes an optical-to-electrical converter 505, a plurality of electrodes 215A, 215B, and one or more electrical-to-optical converters 510A, 510B (also referred to individually or collectively using reference number 510). In other embodiments, the sensing needle 210 includes different, or additional, components than those described in conjunction with FIG. 5. For example, the sensing needle 210 may include various components associated with position tracking with respect to the medical targeting system 140 that are not illustrated in FIG. 5. Further, in some embodiments, various components described below in conjunction with FIG. 5 may be combined, so a single component provides the functionality of multiple components described in conjunction with FIG. 5.

The optical-to-electrical converter 505 is coupled to a fiber optic link 205 that couples between the sensing needle 210 and the controller 230. The optical-to-electrical converter 505 converts an optical signal from the fiber optic link 205 to an electrical signal. For example, the optical-to-electrical converter 505 converts an optical activation signal from the controller 230 to an electrical activation signal. The optical-to-electrical converter 505 is coupled to each of the electrodes 215 of the sensing needle 210 and applies the electrical activation signal to one or more of the plurality of electrodes 215 selected for activation. In an embodiment, the selection of electrodes 215 for activation may be encoded in the optical activation signal. Here, the optical-to-electrical converter 505 may receive the optical activation signal, decode the signal to identify the one or more electrodes 215 for activation, and apply an electrical activation signal to the selected one or more electrodes 215. The optical-to-electrical converter 505 may comprise a photodiode that generates current based on an amount of light incident to the photodiode and absorbed by the photodiode from the fiber optic link 205. In some embodiments, the optical-to-electrical converter 505 further includes a transimpedance amplifier that amplifies current generated by the photodiode and converts the current to a voltage applied to the selected one or more electrodes 215. In some embodiments, the optical-to-electrical converter 505 may receive and convert various types of activation signals that may activate electrodes 215 according to various electrical properties.

The selected one or more electrodes 215 receive the electrical activation signal from the optical-to-electrical converter 505 and generate an electric field that is applied to one or more cells of an anatomy proximate to the sensing needle 210. The electric field may comprise an alternating current (AC) electric field, a direct current (DC) electric field, or a different type of field that may be controlled by the optical activation signal.

Each of the electrodes 215 of the sensing needle 210 are coupled to respective electrical-to-optical converters 510. The electrical-to-optical converters 510 are configured to receive an electrical sensing signal of the respective electrodes 215 that represent electrical characteristics of the cells proximate to the activated electrodes 215. The electrical-to-optical converters 510 converts the electrical sensing signals to one or more optical sensing signals that may be transmitted to the controller 230 via the fiber optic link 205. In some embodiments, the electrical-to-optical converters 510 may include respective amplifiers or other circuitry to amplify or otherwise transform the received signals.

The electrical-to-optical converters 510 may include respective light emitting diode (LED) that output light having controlled intensities controlled by a current from the respective electrodes 215. Here, the potential developed on the electrodes 215 proximate to the tissue being measured may modulate the optical output of the LEDs, thereby encoding the sensed electrical characteristics. In various embodiments, LEDs coupled to different electrodes 215 have different wavelengths (i.e., different colors) to enable light emitted by multiple LEDs to be transmitted to the controller 230 through a single fiber optic channel of the fiber optic link 205. In this example, the controller 230 may apply various color filters to filter the received combined optical signal and recover the signals from individual electrodes 215. In some embodiments, each electrode 215 is coupled to a corresponding electrical-to-optical converter 510 in a one-to-one manner. In other embodiments, the outputs of multiple electrodes 215 may be multiplexed and converted by a shared electrical-to-optical converter 510.

The fiber optic link 205 transmits the optical signals (e.g., activation signals and sensing signals) between the sensing needle 210 and the controller 230. In an embodiment, the fiber optic link 205 may include multiple unidirectional channels. For example, the fiber optic link 205 may include one or more channels for communicating the activation signal to the sensing needle 210 and one or more channels for communicating the sensing signals from the sensing needle 210 to the controller 230. Alternatively, the fiber optic link 205 may include one or more bidirectional channels.

In some embodiments, the electrical connections between components of the sensing needle 210 are generally direct current (DC) connections that do not introduce high frequency noise that may affect the sensing signals.

As described above, although FIG. 5 illustrates the converters 505, 510 as components of the sensing needle 210, the converters 505, 510 could be located in close proximity to the electrode array 220 or at any distance from the tip 240 (e.g., in the tube 200). Furthermore, the optical-to-electrical converter 505 and electrical-to-optical converters 510 could comprise separate devices or could be integrated as a single signal converter.

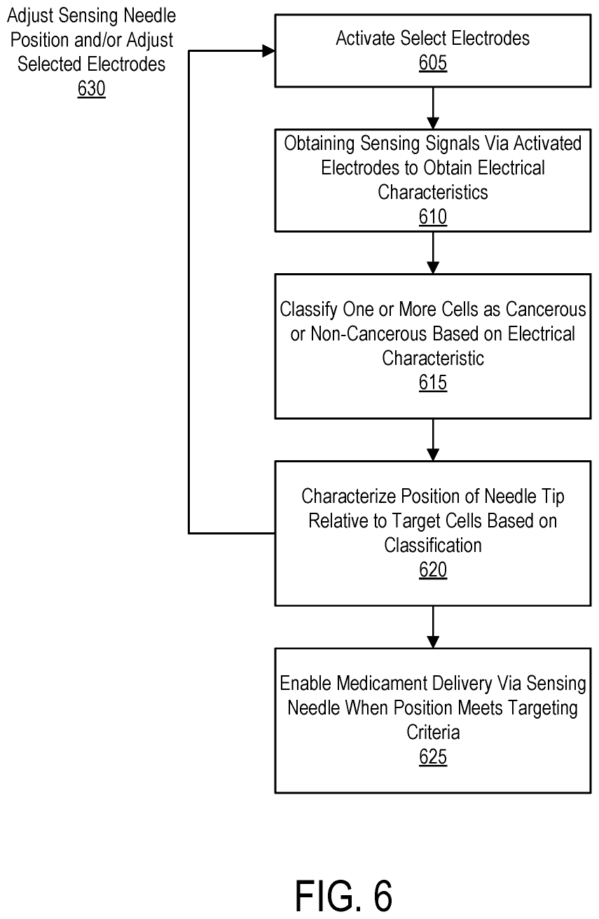

FIG. 6 is a flowchart of one embodiment of a method for controlling medicament delivery. In various embodiments, steps of the method are performed by a tissue classification system 180, as further described above. Further, in various embodiments, the method has fewer or additional steps than those described in conjunction with FIG. 6.

A controller 230 activates 605 one or more electrodes of a sensing needle 210 when the sensing needle 210 is inserted into an anatomy of a patient. In an example operation, a tube 200 and the sensing needle 210 of the tissue classification system 180 is inserted into an anatomical channel of a patient, such as through a trachea and/or bronchial tubes of the patient. Alternatively, the sensing needle 210 may be inserted through tissue. The sensing needle 210 may be manually guided to a target area or may be automatically navigated via a robotic guidance system 120 as described above. The controller 230 activates one or more electrodes 215 to apply an electric field across the one or more cells. The controller 230 may select specific electrodes 215 that receive the activation signal to specify which electrodes 215 are activated. The activation of the electrodes 215 may be controlled by a physician or may be controlled automatically (e.g., by the medical targeting system 140). Different combinations of electrodes 215 may be activated to control various sensing characteristics such as sensing depth, sensitivity, or other characteristics as described above.

The controller 230 obtains 610 sensing signals from the activated electrodes 215 to determine electrical characteristics of cells proximate to the electrodes 215. Based on the sensed electrical characteristic, the controller 230 classifies 615 the one or more cells as cancerous or non-cancerous. In various embodiments, the sensed electrical characteristic is an impedance, and the controller 230 determines a Cole relaxation frequency corresponding to the impedance that may be correlated to a likelihood of cells being cancerous or non-cancerous.

The controller 230 characterizes 620 a position of the sensing needle 210 relative to a target cells based on the classification to determine if position criteria is met. For example, the controller 230 may determine whether or not the sensing needle is sufficiently penetrated into a tumor targeted for treatment. In an embodiment, the relative position may be estimated based on signals received from different electrodes of the sensing needle 210. For example, some electrodes 215 may generate signals indicative of cancerous cells (indicating that those electrodes 215 are in contact with or proximate to the cancerous cells), while other electrodes 215 may generate signals indicative of non-cancerous cells (indicating that those electrodes 215 are not in contact or not sufficiently proximate to the cancerous cells). Based on the layout of the electrodes 215, the controller 230 can characterize, for example, whether the tumor is not penetrated, fully penetrated (i.e., fully surrounding the sensing needle 210), or partially penetrated (i.e., contacting only a portion of the sensing needle 210). The controller 230 may further estimate a depth of penetration. For example, the depth of penetration may be indicative by the number of electrodes 215 near the tip 240 of the sensing needle 210 that detect cancerous cells and the spacing between those electrodes 215. Furthermore, if electrodes 215 on only one side of the sensing needle 210 indicate detection of cancerous cells while electrodes on the other side indicate detection of non-cancerous cells, this may indicate that the sensing needle 210 is adjacent to, but not directly penetrating the target tumor.

The controller 230 may iteratively repeat the activation 605, sensing 610, classification 615, and position characterization 620 steps as the position of the sensing needle 210 and/or the selection of activated electrodes 215 are adjusted 630.

Once desired position criteria is met, the controller 230 may enable 625 medicament delivery via the sensing needle 210. For example, the targeting position criteria may be met when the sensing needle 210 fully penetrates the target tumor with a least a threshold penetration depth such that the medicament can be delivered directly into the target tumor. Enabling medicament delivery may comprise, for example, activating a pump to pump the medicament through the tube 200 and needle tip 240 into the target tumor. Alternatively, enabling the medicament delivery may comprise disabling a locking mechanism that prevents pump activation such that delivery can be manually actuated by a physician. The controller 230 may furthermore generate alerts (e.g., via the I/O device 170 to indicate when the pump is locked to prevent medicament delivery and when it is unlocked or directly activated.

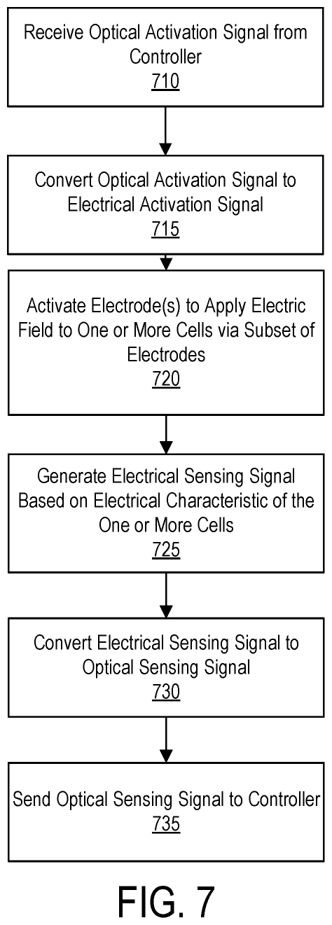

FIG. 7 illustrates an example embodiment of a process for operating a sensing needle using optical activation signals. When the electrodes 215 are appropriately positioned for sensing, the tissue classification system 180 generates and the sensing needle 210 receives 710 an activation signal that is transmitted to the sensing needle 210 through the fiber optic link 205. The sensing needle 210 (or an external signal converter) converts 715 the optical activation signal to an electrical activation signal. In response to the electrical activation signal from the optical-to-electrical converter 505, one or more electrodes 215 are activated 720 to generate an electric field applied to the one or more cells. The controller 230 may select specific electrodes 215 for activating and may control the shape of the electrical field by selecting various combinations of electrodes.

Electrodes 215 of the sensing needle 210 generate 725 an electrical sensing signal (e.g., representing a sensed impedance or other characteristic) based on an electrical characteristic of one or more cells contacting one or more of the electrodes 215. The sensing needle 210 converts 730 the electrical sensing signal to an optical sensing signal. The sensing needle then sends 735 the optical sensing signal from the sensing needle 210 to the controller 230 through the fiber optic link 205.

The described system beneficially enables measurement of electrical characteristics of cells for tissue classification while avoiding significant parasitic effects that may be caused by lengthy electrical connections between the controller 230 and the sensing needle 210. Further, the described system 180 may generate accurate measurements in the presence of bending or twisting of the tube 200. Thus, robust measurement and classification may be achieved.

In various alternative embodiments, the above-described tissue classification system 180 may instead be embodied as a probe device that does not necessarily have a needle tip 240 and is not necessarily configured for dispensing medicament. Such a probe device may be used, for example, to detect a location of a tumor, which may then optionally be treated using an independent medicament delivery device.

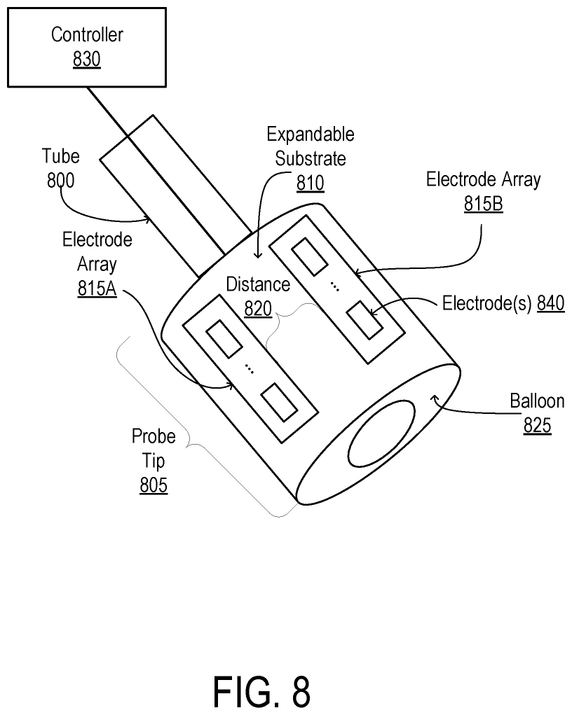

FIG. 8 illustrates one such embodiment of a probe tip 805 for sensing cancerous cells. In this embodiment, the probe tip 805 includes an expandable substrate 810 with a set of electrode arrays 815A, 815B (also referred to individually and collectively using reference number 815) coupled to an external surface of the expandable substrate 810. For purposes of illustration, FIG. 8 shows two electrode arrays 815A, 815B coupled to the expandable substrate 810; however, the number of electrode arrays 815 may vary in different embodiments. Adjacent electrode arrays 815 are separated by a distance 820 (comprising a non-conducting material of the expandable substrate 810). The distance 820 may be uniform between different pairs of electrode arrays 815 or may be non-uniform. In the example of FIG. 8, the electrode arrays 815A, 815B are longitudinally oriented along a length of the probe tip 805 (i.e., electrodes 840 in each electrode array 815 are arranged in longitudinal columns), and the distance 820 between adjacent electrode arrays 815A, 815B is a radial distance.

The probe tip 805 in this example embodiment also includes a balloon 825 or other expandable device. The expandable substrate 810 is coupled to an exterior surface of the balloon 825. The balloon 825 is configured to be inflated or deflated in response to one or more control signals. Inflating the balloon 825 may cause the surface of the expandable substrate 810 to expand in order to reduce proximity of the electrode arrays 815 to the tissue of interest. For example, expanding the balloon 825 may cause the electrode arrays 815 to make and/or maintain conformal contact (or otherwise reduce a gap) with one or more cells of an anatomy proximate to the probe tip 805. Reducing a gap between the electrode arrays 815 and the tissue of interest may reduce degradation of an electric field applied to the cells and enables measurements that mitigates noise introduced from gaps between the electrodes 840 and the tissue. A control signal from the controller 830 causes inflation or deflation of the balloon 825 in various embodiments, with a value of the control signal specifying an amount of inflation or deflation of the balloon 825. While FIG. 8 depicts an embodiment where a balloon 825 is used, in other embodiments, other mechanical systems capable of expanding or contracting the substrate 810 in response to control signals may instead be included in the probe tip 805.

In various embodiments, the electrodes 840 within each electrode array 815 are configured so that the respective distances between adjacent electrodes 840 in the same electrode array 815 remains substantially fixed (i.e., less than a threshold change in distance) regardless of the inflation state of the balloon 825. For example, in the structure of FIG. 8, the expandable substrate 810 may expand radially upon inflation, thus displacing the electrode arrays 816 outwardly and increasing the radial distance 820 between the arrays 815. However, the longitudinal distances between the electrodes 840 within each array 815 may remain substantially the same. In an embodiment, each electrode array 815 may comprise a substantially rigid material that be uniformly displaced relative to other arrays 815 but does not itself expand or contract with the balloon 825. In other embodiments, the electrodes 840 of the electrode arrays 815 comprise a conductive material capable of being expanded in one or more directions without altering their conductive characteristics or otherwise affecting electrical measurements.

The controller 830 is coupled to each electrode array 815 and is configured to communicate an activation signal to at least a subset of the electrodes 840 in one or more electrode arrays 815. As described above, the controller 830 may generate different types of activation signals that cause generation of one or more fields having different characteristics. The controller 830 may furthermore generate one or more control signals to the balloon 825 for controlling inflating and/or deflating. The control signal may specify an amount by which the balloon 825 inflates or deflates. In some embodiments, the control signal specifies a specific portion of the balloon 825 that is inflated, allowing different portions of the balloon 825 to be differently inflated or deflated. Furthermore, the balloon may be designed to enable expansion in a unidirectional manner (e.g., expanding radially without expanding longitudinally to avoid changes in spacing between electrodes 840 within an electrode array 815). As described above, the controller 830 receives sensing signals from the electrode arrays 815 and may classify cells as cancerous or non-cancerous based on sensed electrical characteristics.

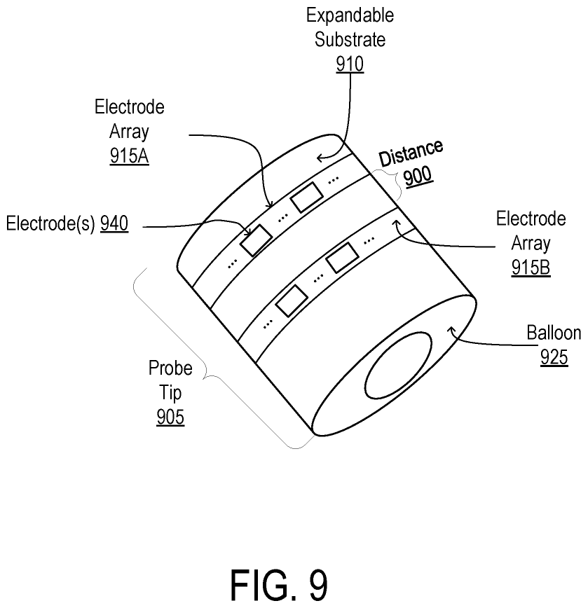

FIG. 9 shows another example probe tip 905 where the set of electrode arrays 915 (e.g., arrays 915A, 915B) are radially oriented on the external surface of the expandable substrate 910. In this example, the distance 900 between adjacent electrode arrays 915A, 915B represents a longitudinal distance. While FIG. 9 shows two electrode arrays 915A, 915B, the set of electrode arrays 815 can include different numbers of electrode arrays 915 positioned along the probe tip 905 in various embodiments. The distance 900 longitudinally separating adjacent electrode arrays 915 may be constant for each pair of adjacent electrodes arrays 915, or may vary. In this example embodiment, the spacing between electrodes 940 in each array 915 may be fixed in the radial direction. Inflation of the balloon 925 may cause longitudinal expansion of the substrate 810 (which may change the distance 900 between arrays 915) without substantially affecting radial spacing of the electrodes 940 within each array 915.

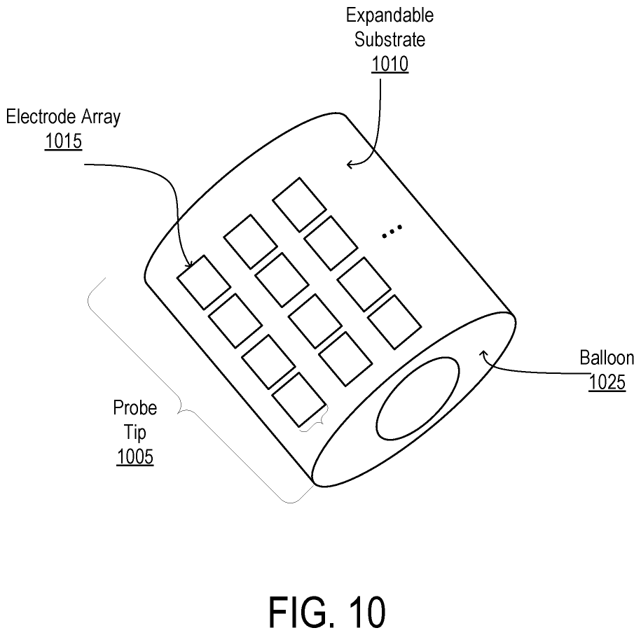

FIG. 10 shows another example embodiment of a probe tip 1005. In this example, the probe tip 1005 includes electrode arrays 1015 arranged both longitudinally and radially (e.g., in a matrix of electrode arrays 1015). Each electrode array 1015 may include electrodes 1040 arranged longitudinally, radially, or in a matrix. Different combinations of electrodes 1040 may be activated to modulate the electrical field (or other fields) to characterize tissue proximate to the probe tip 1005. For example, the controller 1030 may employ a sweeping technique to sweep a volume of interest with different localized fields and measure respective electrical characteristics. The individual measurements may be compared with an average signal to identify inhomogeneity. Electrodes 1040 in the area of interest may then be activated to obtain more localized characterization of the cells.

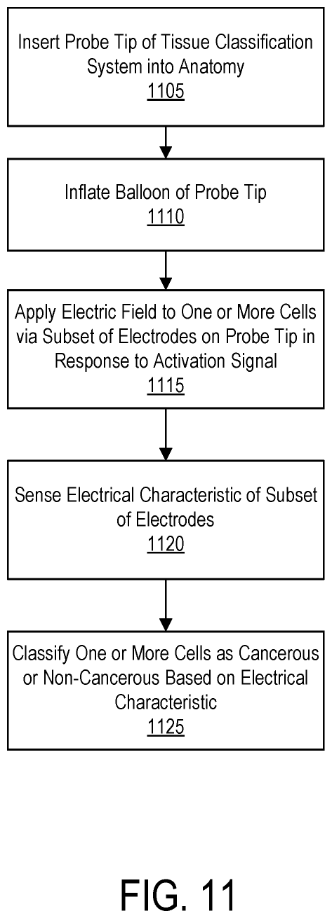

FIG. 11 is a flowchart of one embodiment of a method for classifying one or more cells as cancerous or non-cancerous using a probe tip with an expandable substrate. A probe tip 805 of a tissue classification system 180 is inserted 1105 into an anatomy of a patient. For example, a tube 800 and the probe tip 805 of the tissue classification system 180 is inserted 1105 into an anatomical channel of a patient, such as through a trachea and/or bronchial tubes of the patient. The tube 800 is advanced so at least a portion of the probe tip 805 directly contacts (or is closely proximate to) one or more cells. The tissue classification system 180 controls 1110 inflation of a balloon 825 of the probe tip 805 to facilitate contact between electrodes 840 and the one or more cells. In various embodiments, the balloon 825 is configured so portions of the balloon 825 are inflated, while other portions of the balloon 825 remain deflated, allowing the probe tip 805 to conform to a surface of cells to be probed. Spacing between at least some of the activated electrodes 840 in an array 815 may remain fixed regardless of the inflation state, or the electrodes 840 may be configured such that their electrical properties do not change with expansion and contraction.

A controller 830 activates one or more electrodes 840 to apply 1115 an electric field across the one or more cells. The controller 830 may select specific electrodes 815 that receive the activation signal to specify which electrodes 840 are activated. The controller 830 may activate different combinations of electrodes 840 at different times to facilitate various measurements of the cells.

A sensing device 835 senses 1120 an electrical characteristic of a subset of the electrodes 840. Based on the sensed electrical characteristic, the controller 830 classifies 1125 the one or more cells as cancerous or non-cancerous. In various embodiments, the sensed electrical characteristic is an impedance, and the controller 830 determines a Cole relaxation frequency corresponding to the impedance that may be correlated to a likelihood of cells being cancerous or non-cancerous.

While the process of FIG. 11 is described with respect to the embodiment of FIG. 8, the process may similarly be applied by the embodiments of FIGS. 9-10, or other embodiments of a sensing probe tip with an expandable substrate.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A tissue classification system for medicament delivery, comprising:
a tube for inserting into an anatomy;
a sensing needle at a distal end of the tube, the sensing needle including:
a first set of electrodes that are selectively activated in response to an activation signal to generate a first electric field that passes through a first group of cells of the anatomy, the first set of electrodes to generate a first sensing signal representing a sensed electrical characteristic of the first group of cells in response to the first electric field; and
a controller configured to apply the activation signal, to receive the first sensing signal, to obtain a first classification of the first group of cells as cancerous or non-cancerous based on the sensed electrical characteristic in the first sensing signal, and to control medicament delivery via the sensing needle based on at least the first classification.

2. The tissue classification system of claim 1, further comprising a second set of electrodes to generate a second electric field that passes through a second group of cells of the anatomy, the second set of electrodes to generate a second sensing signal, wherein the controller furthermore obtains a second classification of the second group of cells based on the second sensing signal, and wherein the controller controls the medicament delivery further based on the second classification.

3. The tissue classification system of claim 2, wherein the first set of electrodes and the second set of electrodes comprise respective radial arrays of electrodes at different distances from a tip of the sensing needle.

4. The tissue classification system of claim 3, wherein the controller controls the medicament delivery by enabling a medicament delivery pump responsive to the first classification and the second classification being indicative of the sensing needle penetrating a target tumor with a predefined penetration depth.

5. The tissue classification system of claim 2, wherein the first set of electrodes and the second set of electrodes are radially spaced on a surface of the sensing needle, and wherein the controller controls the medicament delivery by enabling a medicament delivery pump responsive to both the first classification and second classification classifying the first group of cells and the second group of cells respectively as cancerous.

6. The tissue classification system of claim 1, wherein the controller activates a lockout mechanism of a medicament delivery pump to prevent the medicament delivery via the sensing needle in absence of a delivery criteria based at least in part on the first classification being met.

7. The tissue classification system of claim 1, wherein the controller obtains the first classification by:
determining a Cole relaxation frequency from the sensed electrical characteristic of the first group of cells; and
classifying the first group of cells as cancerous in response to the Cole relaxation frequency being within a specified range of frequencies or classifying the first group of cells as non-cancerous in response to the Cole relaxation frequency being outside the specified range of frequencies.

8. The tissue classification system of claim 1,
wherein the controller is configured to generate the activation signal as an optical activation signal and to receive the first sensing signal as an optical sensing signal, wherein the sensing needle further comprises:
an optical-to-electrical converter to convert the optical activation signal to an electrical activation signal applied to the first set of electrodes; and
an electrical-to-optical converter to convert an electrical sensing signal to the optical sensing signal for outputting to the controller; and
wherein the tube comprises a fiber optic link for communicating the optical activation signal and the optical sensing signal.

9. The tissue classification system of claim 8, wherein the fiber optic link includes:
a first fiber optic channel for communicating the optical activation signal; and
a second fiber optic channel for communicating the optical sensing signal.

10. The tissue classification system of claim 8, wherein the fiber optic link includes a shared fiber optic channel for bidirectionally communicating the optical activation signal and the optical sensing signal.

11. A method for controlling medicament delivery based on tissue classification, the method comprising:
generating a first activation signal to activate at least a first set of electrodes of a sensing needle to cause the first set of electrodes to generate a first electric field that passes through a first group of cells of an anatomy;
obtaining, from the first set of electrodes, a first sensing signal representing a sensed electrical characteristic of the first group of cells in response to the first electric field;
obtaining, based on the first sensing signal, a first classification of the first group of cells as cancerous or non-cancerous;
determining if the sensing needle meets position criteria relative to a target tumor based at least in part on the first classification; and
controlling a medicament delivery system to enable medicament delivery via the sensing needle in response to the sensing needle meeting the position criteria.

12. The method of claim 11, wherein controlling the medicament delivery system comprises enabling a medicament delivery pump.

13. The method of claim 11, further comprising locking a medicament delivery pump responsive to the sensing needle not meeting the position criteria.

14. The method of claim 11, further comprising:
generating a second activation signal to activate at least a second set of electrodes of the sensing needle to cause the second set of electrodes to generate a second electric field that passes through a second group of cells of the anatomy;
obtaining, from a second set of electrodes, a second sensing signal representing a sensed electrical characteristic of the second group of cells in response to a second electric field;
obtaining, based on the second sensing signal, a second classification of the second group of cells as cancerous or non-cancerous; and
wherein determining if the sensing needle meets position criteria relative to a target tumor is further based at least in part on the second classification.

15. The method of claim 14, wherein the first set of electrodes and the second set of electrodes comprise respective radial arrays of electrodes at different distances from a tip of the sensing needle, and wherein determining if the sensing needle meets the position criteria comprises:
determining, based on at least the first classification and the second classification, that the sensing needle achieves at least a predefined penetration depth into the target tumor.

16. The method of claim 14, wherein the first set of electrodes and the second set of electrodes are radially spaced on a surface of the sensing needle, and wherein determining if the sensing needle meets the position criteria comprises:
determining, based on at least the first classification and the second classification, that the sensing needle is fully surrounded by the target tumor.

17. The method of claim 11, wherein obtaining the first classification comprises:
determining a Cole relaxation frequency from the sensed electrical characteristic of the first group of cells; and
classifying the first group of cells as cancerous in response to the Cole relaxation frequency being within a specified range of frequencies or classifying the first group of cells as non-cancerous in response to the Cole relaxation frequency being outside the specified range of frequencies.

18. The method of claim 11,
wherein generating the first activation signal comprises:
generating an optical activation signal at a controller;
transmitting the optical activation signal over a fiber optic link coupled between the controller and the sensing needle;
converting the optical activation signal to an electrical activation signal by an optical-to-electrical converter of the sensing needle;
and wherein obtaining the first sensing signal comprises:
generating an optical sensing signal by an electrical-to-optical converter of the sensing needle based on an electrical sensing signal; and
transmitting the optical sensing signal over the fiber optic link from the sensing needle to the controller.

19. A medical targeting system for controlling medicament delivery to a target tumor based on tissue classification, the medical targeting system comprising:
an imaging system including an endoscope for capturing images of an anatomy;
a tissue classification system for controlling medicament delivery, including:
a tube for inserting into an anatomy;
a sensing needle at a distal end of the tube, the sensing needle including:
a first set of electrodes that are selectively activated in response to an activation signal to generate a first electric field that passes through a first group of cells of the anatomy, the first set of electrodes to generate a first sensing signal representing a sensed electrical characteristic of the first group of cells in response to the first electric field; and
a controller configured to apply the activation signal, to receive the first sensing signal, to obtain a first classification of the first group of cells as cancerous or non-cancerous based on the first sensing signal, and to control medicament delivery via the sensing needle based on at least the first classification.

20. The medical targeting system of claim 19, further comprising:
a robotic guidance system to control navigation of at least one of the endoscope and the sensing needle.
